(19) United States
(12) Patent Application Publication
(10) Pub. No.: US 2025/0117884 A1
(43) Pub. Date: Apr. 10, 2025

(54) METHOD AND SYSTEM FOR THE CORRECTION OF ARTIFACTS IN OPTICAL COHERENCE TOMOGRAPHY (OCT) IMAGES

(71) Applicant: Cambridge Enterprise Ltd, Cambridge (GB)

(72) Inventors: Benn Jessney, St Neots (GB); Xu Chen, Cambridge (GB); Michael Roberts, Cambridge (GB); Martin Bennett, Cambridge (GB)

(21) Appl. No.: 18/481,381

(22) Filed: Oct. 5, 2023

(51) Int. Cl. G06T 5/10; A61B 5/00; G06T 5/20; G06T 5/50; G06T 7/11; G06T 11/00; G16H 30/40

(52) U.S. Cl. CPC G06T 5/10; A61B 5/0044; A61B 5/0066; G06T 5/20; G06T 5/50; G06T 7/11; G06T 11/006; G16H 30/40; G06T 2207/10024; G06T 2207/10101; G06T 2207/20021; G06T 2207/20048; G06T 2207/20081; G06T 2207/20084; G06T 2207/20221; G06T 2207/30048; G06T 2207/30101; G06T 2210/41; G06T 2211/441; G06T 2211/456

(57) ABSTRACT

Disclosed is a method (100) for correcting artifacts in Optical Coherence Tomography (OCT) images. The method involves acquiring an OCT image and subsequently transforming it from a Cartesian coordinate system to a polar coordinate system via polar reconstruction, generating a reconstructed polar OCT image. This image is then segmented into RED, GREEN, BLUE color channels. Fourier transformation is applied to each channel, transitioning them into their frequency domain representations. A custom frequency mask is utilized on these images, filtering out artifact-related frequencies. Following this, an inverse Fourier transformation is executed, reverting the images back to their Cartesian format. These images are then combined to produce a reconstituted OCT image. Further, this reconstituted image is merged with the original OCT image, resulting in an OCT image that is substantially free from artifacts.

### TECHNICAL FIELD

The present disclosure relates to a method for the correction of artifacts in Optical Coherence Tomography (OCT) images. Moreover, the present disclosure relates to a system for the correction of artifacts in OCT images.

### BACKGROUND

Coronary artery disease (CAD) remains a leading cause of morbidity and mortality worldwide. CAD is characterized by the buildup of plaque inside coronary arteries, which can lead to blockages and reduced blood flow to the heart. This, in turn, can result in heart attacks and other severe cardiovascular events. Early and accurate diagnosis, as well as the monitoring of CAD, is crucial for effective treatment and prevention of further complications. Advanced atherosclerotic coronary plaques, including thin-cap fibroatheromas (TCFAs), are responsible for the majority of myocardial infarctions. Thus, imaging modalities that can identify TCFA and other higher risk plaques features in vivo are of considerable importance.

In recent years, Optical Coherence Tomography (OCT) has emerged as a preferred imaging technique for studying CAD. OCT offers high-resolution cross-sectional images of the coronary arteries, providing clinicians with detailed views of the artery wall, plaque build-up, and other pertinent features. In particular, OCT can both identify different tissues, including normal vessels, lipid and calcium, and measure both lipid and calcium arc and depth as well as micron-level features such as fibrous cap thickness. Different plaque features identified by OCT are predictive of multiple adverse cardiovascular events (MACE), including plaque burden, lipid arc, fibrous cap thickness and presence of macrophages. These properties mean that OCT can help guide stent placement, identify lesions at higher risk of causing future events, and monitor the efficacy of drugs that may reduce atherosclerotic plaque size or promote plaque stability.

However, as with many imaging modalities, OCT is not without its challenges. One of the primary issues associated with OCT imaging of coronary arteries is the presence of artifacts in the images. These artifacts, which can arise due to various reasons related to the acquisition technique or inherent properties of the imaging modality, compromise the clarity and accuracy of OCT images. The range of image artifacts inherent to OCT technology and associated techniques can limit its usefulness both clinically and for research. For example, although OCT provides incremental prognostic information and has a high negative predictive value, studies show a high prevalence of vulnerable plaque features but a low positive predictive value. Published consensus standards rightly recommend that measurements should be made on good-quality images that do not contain artifacts, but this potentially excludes a significant number of frames containing a large amount of clinical data from investigation.

Such artifacts can lead to potential misinterpretations and, consequently, suboptimal clinical decisions. The true burden of artifacts is difficult to judge, with some studies reporting up to 11.2% of OCT frames being unusable, or having strict eligibility criteria that include a continuous arc of at least 270° around the lumen center and a qualitative definition of the superficial plaque components. Many artifacts are caused by strong scattering or the attenuation of the light beam that reduces its penetration into the arterial wall, resulting in signal-poor areas where there is insufficient information for accurate clinical measurements or tissue classification. However, it is not known whether pre-processing OCT images can increase the proportion of frames available for analysis without affecting the measurements of higher-risk features.

In conventional practice, when faced with OCT images having artifacts, clinicians often resort to ignoring those frames or abstaining from taking measurements from them. This approach, albeit practical, results in a significant loss of potentially valuable information. Each discarded frame represents a missed opportunity for understanding the patient's condition better and making more informed treatment decisions. Moreover, manually identifying artifacts and which frames to disregard introduces subjectivity and inconsistency into the diagnostic process. Different clinicians might have varying thresholds for what they consider to be an “unusable” frame, leading to discrepancies in diagnosis and treatment recommendations.

Addressing artifacts in OCT images is not merely about identifying and removing unwanted features. The true challenge lies in distinguishing between genuine anatomical structures of the coronary artery and artifacts that look similar. Moreover, it's essential to ensure that in the process of correcting artifacts, the underlying anatomy of the artery and plaque is not altered or lost. Another challenge pertains to the variability of artifacts. These can range from shadows caused by thrombus or macrophages, to issues like inadequate flushing and the presence of gas bubbles. Each artifact type may have its unique characteristics and might affect the OCT image differently. Hence, a one-size-fits-all approach to artifact correction is unlikely to be effective.

Some attempts have been made to address the problem of artifacts in OCT images of coronary arteries. However, most of these solutions have been hardware-based, focusing on refining the OCT equipment or altering the acquisition technique. While these hardware-centric approaches have yielded some improvements, they have not been able to eliminate the problem of artifacts entirely. Furthermore, such solutions often come with increased costs, complexity, and may require extensive training for the medical staff.

Therefore, in light of the foregoing discussion, there exists a need to overcome the aforementioned limitations/drawbacks.

### SUMMARY

The aim of the present disclosure is to provide a method and a system to effectively mitigate the impact of artifacts in OCT images of coronary arteries, enhancing the clarity and accuracy of the OCT images while preserving the underlying anatomy of the artery and plaque, thus ensuring that no critical information is lost or distorted in the process. The aim of the present disclosure is achieved by a method and a system for the correction of artifacts in OCT images as defined in the appended independent claims to which reference is made to. Advantageous features are set out in the appended dependent claims.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

FIG. 1 is a process flow for artifact correction, in accordance with one or more embodiments of the present disclosure;

FIG. 2 is an outline of a study for artifact correction, in accordance with one or more embodiments of the present disclosure;

FIG. 3A provides comparison of an uncorrected OCT image and an artifact-corrected OCT image, in accordance with one or more embodiments of the present disclosure;

FIG. 3B provides a graph depicting the effect of correction on different types of artifact, in accordance with one or more embodiments of the present disclosure;

FIG. 4A provides minimum lumen diameter measurements taken on uncorrected OCT images, in accordance with one or more embodiments of the present disclosure;

FIG. 4B provides minimum lumen diameter measurements taken on artifact-corrected OCT images, in accordance with one or more embodiments of the present disclosure;

FIG. 4C provides correlation coefficient plots for lumen area, minimum and maximum lumen diameter measurements, in accordance with one or more embodiments of the present disclosure;

FIG. 4D provides a Bland-Altman plot showing the agreement between minimum lumen diameter measurements taken on uncorrected and artifact-corrected OCT images, in accordance with one or more embodiments of the present disclosure;

FIG. 5A provides a fibrous cap thickness (FCT) measured on uncorrected OCT images, in accordance with an embodiment of the present disclosure;

FIG. 5B provides an FCT measured on artifact-corrected OCT images, in accordance with an embodiment of the present disclosure;

FIG. 5C provides an FCT measured on co-registered histology images, in accordance with an embodiment of the present disclosure;

FIG. 5D provides a Bland-Altman plot of FCT of artifact-corrected OCT images and histology images, in accordance with an embodiment of the present disclosure;

FIG. 5E provides a lipid arc measured on uncorrected OCT images, in accordance with an embodiment of the present disclosure;

FIG. 5F provides a lipid arc measured on artifact-corrected OCT images, in accordance with an embodiment of the present disclosure;

FIG. 5G provides a lipid arc measured on co-registered histology images, in accordance with an embodiment of the present disclosure;

FIG. 5H provides a Bland-Altman plot of artifact-corrected OCT images and histology images, in accordance with an embodiment of the present disclosure;

FIG. 6A provides a calcium angle measured on uncorrected OCT images, in accordance with an embodiment of the present disclosure;

FIG. 6B provides a calcium angle measured on artifact-corrected OCT images, in accordance with an embodiment of the present disclosure;

FIG. 6C provides a calcium angle measured on co-registered histology images, in accordance with an embodiment of the present disclosure;

FIG. 6D provides a Bland-Altman plot of calcium arc measurements on corrected OCT images and histology images, in accordance with an embodiment of the present disclosure;

FIG. 7A provides an FCT and lipid arc measured on uncorrected OCT images, in accordance with an embodiment of the present disclosure;

FIG. 7B provides an FCT and lipid arc measured on artifact-corrected OCT images, in accordance with an embodiment of the present disclosure;

FIG. 7C provides a Bland-Altman plot of FCT measurements on artifact-corrected OCT images compared with uncorrected OCT images, in accordance with an embodiment of the present disclosure;

FIG. 7D provides a Bland-Altman plot of lipid arc measurements on artifact-corrected OCT images compared with uncorrected OCT images, in accordance with an embodiment of the present disclosure;

FIG. 8 is a flowchart listing steps involved in a method for the correction of artifacts in Optical Coherence Tomography (OCT) images, in accordance with an embodiment of the present disclosure; and

FIG. 9 is a schematic block diagram of a system for the correction of artifacts in Optical Coherence Tomography (OCT) images, in accordance with an embodiment of the present disclosure.

### DETAILED DESCRIPTION OF EMBODIMENTS

The following detailed description illustrates the embodiments of the present disclosure and the ways in which they can be implemented. Although some modes of carrying out the present disclosure have been disclosed, those skilled in the art would recognize that other embodiments for carrying out or practicing the present disclosure are also possible.

In a first aspect, the present disclosure provides a method for correction of artifacts in Optical Coherence Tomography (OCT) images, the method comprising:
acquiring a given OCT image;
transforming, by polar reconstruction, the given OCT image from a Cartesian coordinate system to a polar coordinate system, to generate a reconstructed polar OCT image;
splitting the reconstructed polar OCT image into a Red color channel, a Green color channel and a Blue color channel, to generate corresponding polar coordinate images;
performing a Fourier transform on each of the Red color channel, the Green color channel and the Blue color channel in the corresponding polar coordinate images, to convert the corresponding polar coordinate images to respective frequency domain images;
applying a custom frequency mask to each of the frequency domain images to filter out frequencies corresponding to one or more artifacts, to generate respective corrected frequency domain images;
performing an inverse Fourier transform on each of the corrected frequency domain images, to generate respective output images in the Cartesian coordinate system, with one output image for each of the Red color channel, the Green color channel and the Blue color channel;
combining the output images, from each of the Red color channel, the Green color channel and the Blue color channel, to generate a reconstituted OCT image; and
merging the reconstituted OCT image with the given OCT image, to generate an artifact-corrected OCT image.

The present method offers a systematic and comprehensive approach to correcting artifacts in the OCT images. By transforming the image from Cartesian to polar coordinates, it effectively targets radial artifacts common in artery imaging. Individual processing of RGB channels enables precision, addressing unique artifact characteristics in each channel. Using Fourier transformation, the method isolates and corrects artifacts in the frequency domain, ensuring genuine anatomical details remain undistorted. The custom frequency mask is utilized to selectively alter artifact-associated frequencies. Reconstituting the RGB channels retains the image's full-color depth, and merging the corrected OCT image with the original ensures preservation of all important details. Overall, this synergistic approach ensures clearer, artifact-free, and clinically valuable OCT images.

In a second aspect, the present disclosure provides a system for the correction of artifacts in Optical Coherence Tomography (OCT) images, the method comprising:
an input module configured to acquire a given OCT image; and
a processing module in signal communication with the input module, the processing module configured to:
   transform, by polar reconstruction, the given OCT image from a Cartesian coordinate system to a polar coordinate system, to generate a reconstructed polar OCT image;
   split the reconstructed polar OCT image into a Red color channel, a Green color channel and a Blue color channel, to generate corresponding polar coordinate images;
   perform a Fourier transform on each of the Red color channel, the Green color channel and the Blue color channel in the corresponding polar coordinate images, to convert the corresponding polar coordinate images to respective frequency domain images;
   apply a custom frequency mask to each of the frequency domain images to filter out frequencies corresponding to one or more artifacts, to generate respective corrected frequency domain images;
   perform an inverse Fourier transform on each of the corrected frequency domain images, to generate respective output images in the Cartesian coordinate system, with one output image for each of the Red color channel, the Green color channel and the Blue color channel;
   combine the output images, from each of the Red color channel, the Green color channel and the Blue color channel, to generate a reconstituted OCT image; and
   merge the reconstituted OCT image with the given OCT image, to generate an artifact-corrected OCT image.

The present system provides an integrated approach to artifact correction in the OCT images. The system comprises the input module to acquire the OCT image, and the processing module for image transformation and correction. The processor uses polar reconstruction to effectively target radial artifacts by converting the image from Cartesian to polar coordinates. By splitting the image into RGB channels, the processor enables precise corrections for each channel. Through Fourier transformation, the processor identifies and addresses artifacts in the frequency domain, ensuring that the original anatomical details remain clear. Custom frequency masks are applied to enhance the accuracy of artifact removal. After correction, the channels are combined to ensure consistent color depth. The corrected OCT image is then merged with the original, ensuring clarity and detail, resulting in a clean and clinically useful OCT image.

The method comprises acquiring the given OCT image. That is, first, the OCT image is obtained. The term “Optical Coherence Tomography (OCT)” refers to a non-invasive imaging technique that captures high-resolution cross-sectional images of biological tissues. OCT, as an imaging modality, uses light waves to capture micrometer-resolution, cross-sectional images of biological tissues. Acquiring an OCT image involves directing a beam of light onto the tissue of interest and then measuring the echo time delay and intensity of the reflected light. A scanning mechanism then captures multiple such measurements across a specified area, and these measurements are algorithmically processed to construct a two-dimensional or three-dimensional representation of the tissue's internal structure. This acquired image serves as the starting point for the subsequent artifact correction process.

In present embodiments, the given OCT image is of a coronary artery. That is, the given OCT image, acquired through OCT imaging procedures, captures the coronary artery's detailed view, encompassing aspects like the artery wall, plaque formations, and other relevant anatomical features. Due to the presence of various features such as plaques, lumen, and arterial walls, the given OCT image aims to capture these details with the highest possible resolution. In medical imaging, the coronary arteries are of significant interest due to their role in cardiovascular health. The coronary arteries are blood vessels responsible for supplying oxygenated blood to the heart muscle. It may be noted that the intricate nature of coronary arteries, with their winding paths, overlapping structures, and presence of both naturally occurring features and those resulting from medical interventions, often introduces artifacts into OCT images. These artifacts can hinder accurate diagnosis and, in the context of interventions, may affect decisions related to treatment strategies. Given their critical function, any obstructions, anomalies, or diseases affecting these arteries can have severe consequences, leading to conditions such as coronary artery disease (CAD). The specific focus on coronary arteries emphasizes the applicability of the present disclosure for artifact correction in the OCT images of the coronary arteries.

When imaging issues inside the body, and specifically coronary arteries, using OCT, the resultant images frequently present with certain irregularities or distortions, commonly referred to as artifacts. These artifacts emerge due to a myriad of factors, ranging from the inherent complexities of the coronary artery's anatomy to external interventions and even the imaging process itself. Such artifacts, if not addressed, can obscure essential details in the OCT images, potentially leading to challenges in accurate diagnosis and interpretation. The present method allows for correction of such artifacts in OCT images. The following description provides a comprehensive understanding of the method and its individual components.

The method comprises transforming, by polar reconstruction, the given OCT image from the Cartesian coordinate system to the polar coordinate system, to generate the reconstructed polar OCT image. That is, once the OCT image is acquired, it undergoes a transformation process known as polar reconstruction. “Polar reconstruction” refers to the conversion of an image's representation from the Cartesian coordinate system to the polar coordinate system. The Cartesian coordinate system is a two-dimensional coordinate system wherein each point is uniquely determined by a pair of numerical coordinates. In contrast, the polar coordinate system represents points in a plane using a distance from a reference point (usually the origin) and an angle from a reference direction. The process of polar reconstruction, which facilitates this transformation, involves mathematically mapping each point from the Cartesian image to its corresponding location in the polar image. This mapping ensures that all details, contrasts, and features of the given OCT image (original image) are retained in the reconstructed polar image, although represented in a more suitable coordinate system.

This transformational step caters to the unique structural characteristics of the subjects typically imaged using OCT, such as the cylindrical nature of coronary arteries. The rationale behind this transformation is to facilitate easier identification and correction of radial artifacts. By transforming the given OCT image from the Cartesian to the polar coordinate system, the method ensures that the image is represented in a manner that aligns with the inherent geometry of the imaged tissue. Specifically, for coronary arteries, this transformation results in an image where radial features like the vessel lumen, plaque formations, or any other anomalies are represented uniformly and without the distortions that might arise in the Cartesian representation. The result of this transformation is a “reconstructed polar OCT image,” which retains all the information of the original but is represented in the polar coordinate system.

In an embodiment, the method further comprises analyzing the OCT image, before transforming the given OCT image, to detect one or more of a vessel lumen and a guidewire shadow therein by implementing a neural network. The objective of this analysis step is the detection of certain features within the OCT image relevant for coronary arteries, specifically the vessel lumen and the guidewire shadow. The vessel lumen, the innermost open space within a vessel through which blood flows, is an anatomical feature in coronary artery imaging. Accurate identification of the lumen is important for various diagnostic and therapeutic procedures. On the other hand, the guidewire shadow is an artifact that often appears in OCT images, especially when imaging is performed during certain interventional procedures. The guidewire, a medical instrument used to guide catheters or other devices within the vascular system, can introduce shadows or dark regions in the OCT images. These shadows, if not identified, can lead to image misinterpretation, and may thus affect the subsequent artifact correction process.

To achieve the detection of the vessel lumen and the guidewire shadow, the method employs a neural network. Neural networks are a category of algorithms within the broader field of artificial intelligence and machine learning. These algorithms are designed to recognize patterns within data, making them suitable for tasks such as image feature detection.

In an embodiment, the neural network is a deep convolutional network with an encoding-decoding architecture, and wherein the neural network is trained to detect the vessel lumen and/or the guidewire shadow in the OCT images. Herein, the neural network employed for the detection tasks is characterized as a deep convolutional network with the encoding-decoding architecture. Deep convolutional networks, often referred to as Convolutional Neural Networks (CNNs), are a subclass of neural networks particularly tailored for image analysis. These networks utilize convolutional layers to automatically and adaptively learn spatial hierarchies of features from the input images. Given the spatial nature of OCT images, with intricate details and varying contrasts, CNNs are aptly suited for their analysis. The encoding-decoding architecture, often seen in networks designed for tasks like image segmentation, is a two-fold process. The encoding phase involves a series of convolutional layers that progressively reduce the spatial dimensions of the input image while increasing the depth, capturing the essential features and details. Following the encoding phase is the decoding phase, wherein the compressed feature representation is gradually up-sampled to restore the image to its original dimensions, but with the desired features, in this case, the vessel lumen and/or the guidewire shadow, distinctly highlighted or segmented.

In the context of the present method, the neural network is trained on a dataset of OCT images wherein the vessel lumen and guidewire shadow have been previously identified and labeled. Through this training, the neural network learns to discern the patterns and characteristics associated with the vessel lumen and the guidewire shadow. Once adequately trained, the neural network can then process new, unlabelled OCT images and accurately detect these features.

By integrating this neural network-based analysis step, the method ensures that the given OCT image, which will undergo further transformation and artifact correction, is already optimized in terms of the identification of critical features and artifacts. This optimization not only improves the efficiency of the subsequent steps but also enhances the overall accuracy and reliability of the artifact correction process, leading to OCT images that are of superior quality and clinical relevance.

The method further comprises splitting the reconstructed polar OCT image into a Red color channel, a Green color channel and a Blue color channel, to generate the corresponding polar coordinate images. That is, the reconstructed polar OCT image is divided into three distinct color channels: Red, Green, and Blue (often abbreviated as RGB). It may be understood that every digital image, including the reconstructed polar OCT image, is typically composed of a combination of primary color channels: Red, Green, and Blue. These channels collectively represent the entire spectrum of colors seen in the image. However, each channel individually emphasizes varying details and contrasts within the image, capturing distinct information that is sometimes not discernible when all channels are combined. By separating the reconstructed polar OCT image into these discrete color channels, the method facilitates a more granular and targeted approach to image processing. The Red color channel, for instance, may emphasize details and contrasts associated with redder hues in the image, while the Green and Blue color channels will focus on their respective color spectrums. By treating each channel individually, the method can address and correct artifacts that might be more prominent or only present in one specific channel.

Herein, while the general structure of the coronary artery or other imaged tissue remains consistent across all channels, subtle differences in how each channel captures this structure can provide valuable insights. For instance, certain artifacts or tissue features might be more pronounced in the Red channel compared to the Green or Blue channels. By processing each channel separately, the method ensures that such nuances are not overlooked or overshadowed by dominant features from other channels. Herein, once each color channel has been individually processed, the corresponding polar coordinate images for the Red, Green, and Blue channels can be generated. These images, though derived from the same original OCT image, offer different perspectives and layers of detail, emphasizing the importance of dissecting the image into its primary color components.

The method further comprises performing the Fourier transform on each of the Red color channel, the Green color channel and the Blue color channel in the corresponding polar coordinate images, to convert the corresponding polar coordinate images into their respective frequency domain images. This step involves applying the Fourier transform to each of the RGB channels of the polar coordinate images. The term “Fourier transform” denotes a mathematical operation that converts a function (in this case, the image) from its original domain (spatial domain) into a representation in the frequency domain. The Fourier transform is performed in the present method because many artifacts in OCT images manifest as specific frequencies in the frequency domain. By converting the OCT image into this domain, the method can more easily identify and target these artifact-associated frequencies.

In particular, herein, the method involves applying the Fourier transform individually to the Red, Green, and Blue color channels of the polar coordinate images. This ensures a comprehensive breakdown of the entire OCT image. Each color channel, as discussed, captures distinct details and contrasts within the overall image. When transformed to the frequency domain, these channels reveal the different frequencies that dominate their specific color spectrum. For instance, certain artifacts or tissue features might manifest more prominently within a specific frequency range of the Red channel as compared to the Green or Blue channels. In the frequency domain representation, these artifact-associated frequencies become more discernible, distinct from the frequencies representing genuine anatomical details. This distinction helps with precise artifact correction in subsequent steps of the method, as discussed in the following paragraphs.

The method further comprises applying a custom frequency mask to each of the frequency domain images to filter out frequencies corresponding to the one or more artifacts, to generate respective corrected frequency domain images. Upon transformation of the polar coordinate images into the frequency domain, the representation of the OCT image shifts from spatial relationships to a composition based on constituent frequencies. In this frequency domain representation, the various elements of the image, including genuine anatomical structures and unwanted artifacts, are represented as distinct frequency components. Herein, the custom frequency mask is a pre-defined filter tailored to selectively target and alter (attenuate) or entirely remove the frequencies associated with known artifacts. This mask is designed based on analysis of a plurality of OCT images with known artifacts, wherein the frequency signatures of common artifacts, such as guidewire shadows, thrombus, or other inconsistencies, have been identified.

By applying the custom frequency mask to each of the frequency domain images, the method can effectively isolate the influence of these artifact-associated frequencies. It is akin to a precise filtering mechanism where unwanted noise or disturbances are systematically removed from a signal. By operating in the frequency domain, the method leverages the distinct frequency signatures of artifacts, enabling a precise and effective correction mechanism. The end result is the generation of corrected frequency domain images that, when reverted to their spatial counterparts, offer clinicians and researchers OCT images of enhanced clarity, free or nearly-free of common distortions and artifacts, thereby facilitating more accurate diagnosis and analysis (as discussed in the subsequent paragraphs).

In an embodiment, the custom frequency mask is configured to utilize frequency filtering to alter the magnitude of pixels corresponding to the artifacts. It may be understood that when an OCT image is transformed into the frequency domain, every constituent element, be it genuine anatomical structures or undesired artifacts, is represented by certain frequency components. The magnitude of these components, in the context of image processing, is directly related to the pixel intensities in the spatial image. In simple terms, higher magnitudes in the frequency domain correspond to more pronounced or brighter features in the spatial representation, and vice versa. The artifacts in the OCT image, given their nature, have specific frequency signatures, meaning they manifest as distinct frequency components with certain magnitudes in the frequency domain. Now, herein, the custom frequency mask utilizes frequency filtering to alter the magnitude of these specific frequencies. By adjusting the magnitude, the custom frequency mask effectively diminishes the prominence or visibility of the artifact in the spatial image, without entirely erasing its presence. This process of altering magnitude rather than complete removal ensures that while the artifact's impact is minimized, the surrounding genuine anatomical details remain undistorted and intact.

The method further comprises performing an inverse Fourier transform on each of the corrected frequency domain images, to generate the respective output images in the Cartesian coordinate system, with one output image for each of the Red color channel, the Green color channel and the Blue color channel. It may be understood that the images in the frequency domain, after undergoing artifact correction, provide a representation where unwanted frequencies, corresponding to artifacts, have been filtered out. However, for the data to be clinically useful and interpretable, it needs to be presented in a form that depicts spatial relationships; essentially how the different elements of the image relate to each other in space. The inverse Fourier transform serves this purpose by transforming the filtered frequency data back into a spatial representation that forms the given OCT image.

Herein, this transformation is applied individually to each of the corrected frequency domain images corresponding to the primary color channels: Red, Green, and Blue. This channel-by-channel approach ensures that each color component of the OCT image is independently reconstructed. Upon application of the inverse Fourier transform to each channel's corrected frequency domain image, the resultant output images are generated in the Cartesian coordinate system. These individually processed color channel images then form the building blocks for the final, composite OCT image, offering a comprehensive and clear view of the imaged tissue or structure.

The method further comprises combining the output images, from each of the Red color channel, the Green color channel and the Blue color channel, to generate the reconstituted OCT image. It may be considered that while the Red, Green, and Blue color channels were processed separately to address nuances and details specific to each channel, it is required to present an integrated OCT image that offers a comprehensive view of the imaged tissue or structure. This step of reconstitution involves the combination of the output images from each of the three primary color channels: Red, Green, and Blue. These output images, each generated in the Cartesian coordinate system post the application of the inverse Fourier transform, represent the artifact-corrected spatial representations specific to their respective color channels. To generate the reconstituted OCT image, these individual channel images are overlaid and combined in such a manner that the contributions of each channel fuse seamlessly. This ensures that the details captured by each channel are retained in the final image, offering a detailed view that is representative of the imaged tissue.

The method further comprises merging the reconstituted OCT image with the given OCT image, to generate the artifact-corrected OCT image. That is, following the reconstitution of the individual color channels to generate the reconstituted OCT image, the method further includes merging of this reconstituted OCT image with the original, given OCT image. It may be appreciated that the given OCT image, which serves as the initial input for the method, includes both the genuine anatomical details and the undesired artifacts. While the above described steps in the method may help to mitigate these artifacts, it is imperative to ensure that the inherent details of the given OCT image are preserved and not overshadowed by the corrections. To achieve this balance, the reconstituted OCT image, which is the artifact-corrected version derived from the processed color channels, is merged with the given OCT image.

This merging process is not merely an overlay of the two images; but instead, it is a calculated integration that ensures that the refined details from the reconstituted image enhance the genuine anatomical features of the given OCT image, while simultaneously suppressing or eliminating artifacts. The resultant artifact-corrected OCT image retains the details of the given OCT image, while simultaneously incorporating the corrections introduced by the reconstituted OCT image. The resultant artifact-corrected OCT image, thus, offers a clear and unobstructed view of the imaged tissue, facilitating more accurate and informed clinical decision-making.

In an embodiment, the step of merging the reconstituted OCT image with the given OCT image further comprises merging the detected one or more of the vessel lumen and the guidewire shadow, to generate the artifact-corrected OCT image. That is, the process of merging the reconstituted OCT image with the given OCT image is further augmented by the inclusion of the detected features, namely the vessel lumen and the guidewire shadow. Specifically, when merging the reconstituted OCT image with the given OCT image, the detected vessel lumen and guidewire shadow are also integrated. Such integration is done with precision, ensuring that the boundaries and positions of these detected features align accurately with their representations in the given and reconstituted images. This ensures that the final artifact-corrected OCT image is not only refined in terms of reduced artifacts but also include critical anatomical and procedural details.

In an embodiment, the artifact-corrected OCT image comprises corrected artifacts selected from a group consisting of thrombus, macrophage shadows, inadequate flushing, and gas bubbles. That is, the resultant artifact-corrected OCT image represents the refined version where specific, commonly encountered artifacts have been addressed. Each of these artifacts has distinct characteristics and origins. The thrombus, for instance, is a blood clot that can manifest as an irregular mass in the blood vessels, potentially masking critical vessel features. The macrophage shadows, resultant from accumulations of white blood cells, cast dark regions on the image, hiding underlying tissue intricacies. The artifact due to inadequate flushing during certain procedures results in hazy areas in the image due to remnants of blood or other agents. Lastly, gas bubbles introduce bright, distracting spots in the OCT image, often overshadowing finer details. In the artifact-corrected OCT image, the method ensures that each of these prevalent artifacts are addressed, such that the OCT images are reliable and offer clinicians and researchers a more accurate and unobstructed view of the imaged tissue.

Therefore, the present method provides a structured, precise, and comprehensive approach to correcting artifacts in OCT images. By leveraging advanced mathematical transformations, custom frequency masks, and precise processing of RGB channels, the method ensures artifact-free, high-quality OCT images that are important for clinical diagnosis and treatment.

The present disclosure also relates to the system for correction of artifacts in Optical Coherence Tomography (OCT) images as described above. Various embodiments and variants disclosed above, with respect to the aforementioned method, apply mutatis mutandis to the system.

In the system, the input module serves as the primary interface for receiving or acquiring the OCT images. The input module acts as a gateway through which OCT images, generated from various imaging devices or sources, are fed into the system for further processing. The input module may be equipped with capabilities to handle different formats or standards of OCT images, ensuring versatility in accommodating images from various OCT imaging devices. In an example, raw OCT data may be exported in DICOM format as input using an image loader.

Generally, as used herein, the term “processing module” refers to a computational element that is operable to respond to and processes instructions that drive the system. Optionally, the processing module includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processing module” may refer to one or more individual processors, processing devices and various elements associated with a processing device that may be shared by other processing devices. Additionally, the one or more individual processors, processing devices and elements are arranged in various architectures for responding to and processing the instructions that drive the system.

In an embodiment of the system, the given OCT image is of a coronary artery.

In an embodiment of the system, the system further comprises a neural network implemented by the processing module, wherein the neural network is further configured to analyze the OCT image, before transforming the given OCT image, to detect one or more of a vessel lumen and a guidewire shadow therein.

In an embodiment of the system, the processing module, for merging the reconstituted OCT image with the given OCT image, is further configured to merge the detected one or more of the vessel lumen and the guidewire shadow, to generate the artifact-corrected OCT image.

In an embodiment of the system, the neural network is a deep convolutional network with an encoding-decoding architecture, and wherein the neural network is trained to detect the vessel lumen and/or the guidewire shadow in the OCT images.

In an embodiment of the system, the custom frequency mask is configured to utilize frequency filtering to alter the magnitude of pixels corresponding to the artifacts.

The present disclosure also relates to an apparatus comprising a computer program stored in a memory, the computer program being configured to control the apparatus to perform the method as described above. Various embodiments and variants disclosed above, with respect to the aforementioned method and the aforementioned system, apply mutatis mutandis to the apparatus.

The apparatus may be implemented as a standalone workstation that integrates specialized hardware and software components to execute the method efficiently. The apparatus may include a central processing unit (CPU), complemented by a graphics processing unit (GPU) tailored for the intensive image processing tasks, especially beneficial for operations like Fourier transformation and neural network computations. Additionally, the apparatus would feature a dedicated input interface, compatible with various OCT imaging devices, allowing for acquisition of OCT images. Furthermore, to facilitate real-time visualization and assessment, the apparatus may incorporate a display which may render the artifact-corrected OCT images for clinical analysis; or sometimes both the given OCT images and the artifact-corrected OCT images side by side, offering clinicians an immediate comparative view.

The present disclosure also relates to a computer program comprising computer executable program code, when executed the program code controls a computer to perform the method as described above. Various embodiments and variants disclosed above, with respect to the aforementioned method and the aforementioned system, apply mutatis mutandis to the computer program.

The computer program may be implemented as AI-based image pre-processing software that corrects a large proportion of artifacts in the OCT images, increasing the proportion of an OCT pullback that can be assessed. Such OCT image pre-processing corrects a wide range of common artifacts of which a significant proportion would otherwise prevent accurate plaque classification or clinical measurements that would allow risk-stratification. Pre-processing allowed frames which may otherwise be excluded from analysis to be accurately examined. This AI-based pre-processing is non-destructive and retains accuracy of lumen and plaque parameters. These corrected OCT images may assist clinicians and researchers to optimize stent placement and identify higher-risk plaques and the effects of drugs on atherosclerosis.

In light of the existing technological landscape, the present method provides a detailed approach to artifact correction in OCT images, particularly when raw OCT data is not accessible. Unlike the process utilized by existing approaches, which utilizes raw OCT data to correct artifacts before conversion into recognizable colour images, the present method operates on processed colour images, preserving colour by segregating the image into Red, Green, and Blue (RGB) channels. This distinctive approach enables the application of complex Fourier manipulations to each channel independently, which helps to avoid the introduction of further artifacts and excessive tissue smoothing, thus maintaining the recognizability of distinct tissue types in the artifact-corrected OCT image.

Moreover, the utilization of parallel processing channels in the present disclosure provides a technical advancement to address challenges posed by Fourier manipulations. It may be noted that the present method provides a solution to remove artifacts such as gas bubbles and others that may be inherent to the technology post raw data capture, a process not known in existing approaches. The present method, by operating on processed images instead of raw data, broadens its applicability, allowing a diverse range of users, not limited to OCT manufacturers, to correct artifacts in OCT images, making it a versatile and user-friendly solution for OCT image processing. This allows the present method to offer an adaptable solution to a wider audience in the medical imaging community, thereby promoting enhanced accuracy and reliability in OCT image interpretations across varied user profiles.

### Experimental Part

For experimentation, OCT images were examined for the presence of artifacts that prevent accurate tissue identification or plaque measurements. Images were then pre-processed through an artificial-intelligence based neural network for correction as per the embodiments of the present disclosure, and both corrected and uncorrected OCT images compared against an ex-vivo OCT dataset co-registered with histology, and clinical OCT scans.

In particular, the experiment involved examining the range and impact of artifacts inherent to OCT acquisition and those caused by different tissue types. The software was developed to firstly detect artifacts caused by disease and poor acquisition technique and subsequently reconstruct true artifact-free tissue images. Image pre-processing corrected a wide range of common artifacts, allowing more of the whole pullback and dataset to be used in research and real-world clinical practice, and improved plaque classification without affecting the identification of higher-risk plaque features. Thus, the proposed image pre-processing techniques may be a useful adjunct to OCT image analysis.

The results show that image artifacts were present in 48% of OCT frames with 62% of artifacts over or within lesions, preventing assessment and accurate measurement in 12.3% frames of an average pullback. The pre-processing corrected a wide range of common OCT artifacts, including thrombus, macrophage shadows, inadequate flushing, and gas bubbles. Overall, 70% all OCT artifacts were corrected, and true tissue reconstruction achieved in 63% artifacts that would otherwise prevent accurate clinical measurements, reducing unmeasurable frames to 4.6%. Artifact correction was non-destructive and retained anatomical lumen and plaque parameters including lumen areas, diameters, plaque area and burden. Correction improved OCT-based plaque classification compared against histology and retained accurate assessment of higher-risk features.

Thus, it may be concluded that the OCT image pre-processing corrects a wide range of common artifacts allowing more of the whole pullback and dataset to be used in research and real-world clinical practice. AI-based pre-processing can augment OCT image analysis systems both for stent optimization and in natural history studies.

More specifically, for acquisition, raw OCT data was exported in DICOM format as input to the present artifact correction software using an image loader. The Fourier transform was used, which is a technique to decompose an image into its sine and cosine components. The output of the transformation represents the image in the frequency domain, while the input image is the spatial domain equivalent. The number of frequencies corresponds to the number of pixels in the spatial domain image. In the Fourier domain, each point represents a particular frequency contained in the spatial domain image and is obtained by multiplying the spatial image with the corresponding base function (sine and cosine waves with increasing frequencies) and summing the result. In most implementations, the Fourier image is shifted in such a way that the image mean is displayed in the center of the image. The further away from the center an image point is, the higher is its corresponding frequency. Because an image in the Fourier domain is decomposed into its sinusoidal components, certain frequencies of the image may be processed, thus asserting an influence on the image when it is re-transformed to the spatial domain by the inverse Fourier transform.

Once undergoing transformation, it was observed that OCT artifacts show a tendency to appear at consistent points in the frequency domain, with a polar view necessary to maintain this location in the Fourier plot. Utilizing a custom mask, it was possible to utilize frequency filtering to alter the magnitude of pixels belonging to areas of artifact and thus giving the effect of removing artifacts once the image is re-transformed into the spatial domain. In image processing, Fourier transforms are dependent on color information, therefore splitting the image into its component color channels and applying the Fourier transform technique to each channel individually allows a color image to be retained in the final output. The resulting polar image is merged back to a cartesian orientation to be utilized in the output image reconstruction.

To remove OCT artifacts, extensive manipulation of the input image was required. This results in a series of processing artifacts in the lumen and guidewire shadow areas of the OCT image, as these areas contain pixels with similar frequency characteristics to artifacts. The vessel lumen and guidewire shadow were detected utilizing a deep convolutional network with encoding-decoding architecture trained with data from 8 OCT sequences, representing x frames, with lumen contours and guidewire artifact contours manually segmented. Additionally, because of extensive image processing, OCT images that undergo the Fourier transform filtering display tissues with a smoothed appearance which is removed from classical clinical descriptions of plaque OCT characteristics. The final artifact-free output image is therefore a blend of a filtered Fourier transformed image and original lumen, guidewire shadow and original OCT image. The result is an image recognisable to the clinician as an OCT image, with expected plaque characteristics but without artifacts.

This pre-processing software was tested on an ex-vivo dataset. Briefly, following appropriate ethics committee approval and consent from relatives, arteries were harvested from human hearts during autopsy in consultation with a senior pathologist. The left anterior descending artery was dissected and excised along with ˜40 mm of surrounding tissue to maintain overall structural integrity. Side branches were ligated and a guide catheter sutured into the left main stem ostium. A 0.014″ coronary guidewire (either BMW universal or Pilot 50, Abbott Vascular) was advanced, permitting delivery of the intravascular imaging catheter (DragonFly C7, St. Jude Medical). The vessel was fixed to a proprietary designed rig and prewarmed to 37° C. Images were acquired under perfusion pressure at 100 mmHg using 25 mm/s automated pullback before histological processing. All imaging data was stored digitally and exported for offline analysis.

After imaging, arteries were perfusion-fixed in 10% buffered formalin for ≥24 hours. 5 μm sections were cut at 400 μm intervals, maintaining proximal and distal orientation. Sections were stained with haematoxylin-eosin and Van Gieson and reviewed by an independent, experienced cardiac pathologist, blinded to all intracoronary imaging (MG). Sections were given an overall plaque classification and measurements made for lumen dimensions, minimum fibrous cap thickness (FCT), lipid and calcium arcs.

The OCT images were matched to co-registered histological sections by an independent, experienced intravascular imaging investigator, blinded to final histological plaque classification. Detailed measurements were taken during ex-vivo imaging to aid co-registration, and landmarks including bifurcations, guide catheter location, and prominent calcium deposits used for localization.

More specific details of the experimentation are described hereinafter, for reference.

Herein, the clinical dataset was part of The DNA Damage and Repair in Patients with Coronary Artery Disease (DECODE) study, which was a prospective study approved by the Research Ethics Service Committee South East Coast—Surrey, UK (ClinicalTrials.gov NCT02335086). All participants gave written and informed consent before enrolment. Consecutive patients undergoing percutaneous coronary intervention (PCI) for symptomatic stable angina (SA) (n=47) despite optimal medical therapy or NSTEMI (n=42) were prospectively enrolled. All patients underwent OCT intracoronary imaging to determine plaque morphology prior to culprit lesion PCI. A Dragonfly Duo FD-OCT imaging catheter (Abbott Vascular) was used utilizing a 2.7-French monorail delivered through a 6-French guide catheter over a standard 0.014-inch intracoronary guidewire. Imaging data was stored digitally and exported for offline analysis. The clinical dataset comprised consecutive cases with 5 SA and 5 NSTEMI patients contributing 12 OCT sequences. All sequences were analyzed in full for artifacts up to the guide catheter.

Exported OCT frames were pre-processed using proprietary software as per the embodiments of the present disclosure to correct artifacts. Subsequent analysis was performed on uncorrected OCT images and duplicate artifact-corrected OCT images for comparison. Offline OCT analysis was performed using a LightLab Imaging workstation (St. Jude Medical) by an independent observer blinded to histology. Luminal contours were defined, and plaque composition assessed for all image types (uncorrected OCT, corrected OCT and histology). An atherosclerotic plaque was defined as a mass lesion within the arterial wall with loss of the normal tissue tri-layer appearance or focal intimal thickening. Fibrous plaque was defined as a homogenous lesion with high signal attenuation of OCT signal. Calcification was defined as a signal-poor area with sharply delineated borders and low attenuation. Lipid was defined as a signal-poor region with poorly defined borders with fast OCT signal drop-off. Lipid and calcium arc measurements were recorded within each frame. Minimum FCT was measured at its thinnest part 3 times and the mean value was used in subsequent analysis. Measurements of histological sections and imaging data were performed in a random order. A correction factor to account for tissue shrinkage was not applied to histology measurements as arteries were perfusion-fixed and an overall correction factor assumes equal and uniform tissue shrinkage which was felt to be invalid.

Vessel lumen sizes were expressed in millimeters. Tissue measurements were made in microns and arcs in degrees. Agreement between imaging types or with histology was compared using correlation coefficients and a nested ANOVA where appropriate. Comparisons using Bland-Altman plots used a one sample t-test using a threshold for significance as 0.05. A statistically significant result was used to represent proportional bias and therefore potentially an invalid and poor correlation. Statistical analyses were performed in SPSS 28.0.0 (SPSS Inc, IBM Computing).

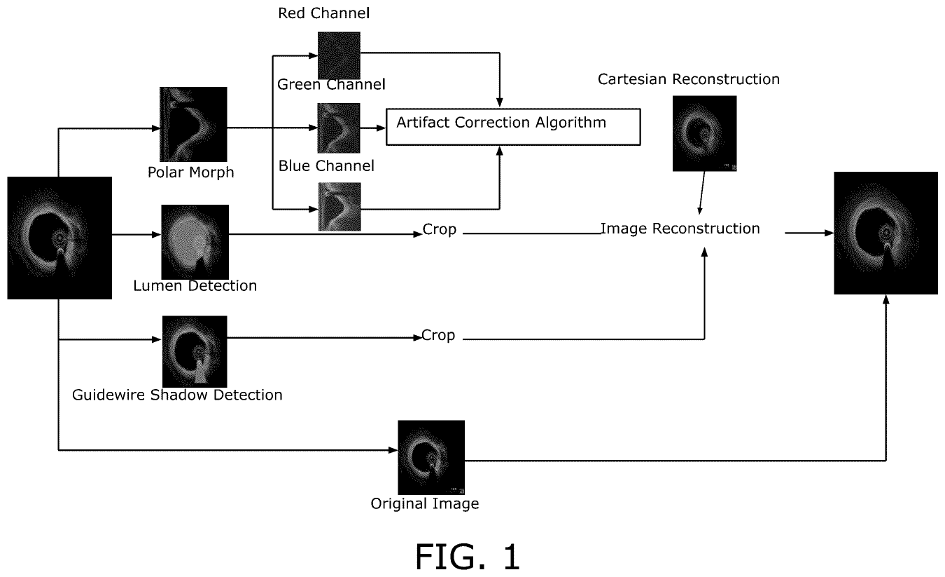

FIG. 1 illustrates a process flow for artifact correction. As illustrated, after image acquisition, data was exported for parallel processing. Firstly, the vessel lumen and guidewire shadow were detected utilizing a deep convolutional network with encoding-decoding architecture. A polar reconstruction was then created from the original OCT image before being split into its respective RGB (red, green, blue) color channels. These images were processed using the proposed techniques to detect artifacts before reconstructing artifact-free tissues. Finally, the output image was reconstructed from outputs of all parallel processing channels. The artifact-corrected OCT sequences were analyzed in parallel to uncorrected OCT runs.

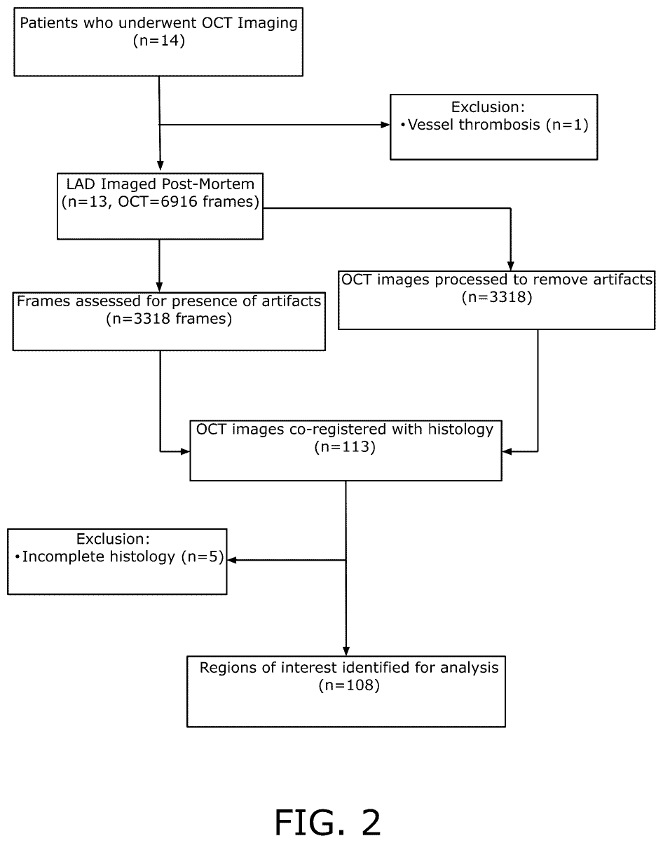

Referring to FIG. 2, illustrated is an outline of a study for artifact correction. Herein, to test the correction processing, first ex vivo OCT pullbacks were analyzed for the presence of artifacts, then images were processed for artifact correction before comparison with co-registered histology. Left anterior descending arteries from 14 human hearts were harvested and underwent OCT imaging under physiological pressures. Occluded vessels were excluded. Patient demographics are presented in Table 1 below. Donors were aged between 47 and 85 years, 71.4% were male, and included both cardiovascular and noncardiovascular causes of death. 6916 frames were analyzed representing 1383 mm of coronary artery.

TABLE 1
Patient demographics

| | | CV Death | Non-CV Death |
|---|---|---|---|
| | Overall (n = 14) | (n = 8) | (n = 6) |
| Male, n (%) | 10 (71.4) | 8 (100.0) | 2 (33.3) |
| Age, y (SD) | 71.1 (11.8) | 76.8 (9.8) | 63.6 (10.3) |

Comorbidities, n (%)

| | | | |
|---|---|---|---|
| Ischemic Heart | 7 (50.0) | 6 (75.0) | 1 (16.7) |
| Disease | | | |
| Cerebrovascular | 2 (14.3) | 1 (12.5) | 1 (16.7) |
| Disease | | | |
| Extracardiac | 6 (42.9) | 4 (50.0) | 2 (33.3) |
| Arteriopathy | | | |
| Diabetes Mellitus | 1 (97.1) | 1 (12.5) | 0 (0.0) |
| Hypertension | 5 (35.7) | 3 (37.5) | 2 (33.3) |
| Cardiac Failure | 6 (42.9) | 4 (50.0) | 2 (33.3) |
| CTEPH | 3 (21.4) | 1 (12.5) | 2 (33.3) |

As per Table 1, CV indicates cardiovascular; and CTEPH indicates chronic 3 thromboembolic pulmonary hypertension.

All 6916 frames were analyzed for artifacts up to the guide catheter, and the most dominant artifact recorded. An artifact was defined as an area of abnormal or poor signal, most frequently due to poor penetration from strong scattering or attenuation, where an accurate clinical measurement could not be taken, or plaque could not be accurately classified. Guidewire shadow, side branch artifacts, and beam divergence were not scored, as guidewire shadow and beam divergence were present in almost all frames. Artifacts confined to the lumen, such as ghost lines caused by reflections in the optical catheter that did not affect the reading of tissues, were also not scored.

An artifact was present in 3318 out of 6916 frames (48.0%), including fold-over artifacts (n=17), gas bubbles (n=804), inadequate flushing (n=510), macrophage shadows (n=325), superficial signal dropout (n=79), saturation (n=38), seam artifacts (n=671), tangential signal dropout (n=295), and thrombus (n=579). Of these, an artifact was over or within a lesion in 2079/3318 frames (62.7%) and prevented accurate clinical measurements in 853/6916 frames or 12.3% in a typical OCT pullback.

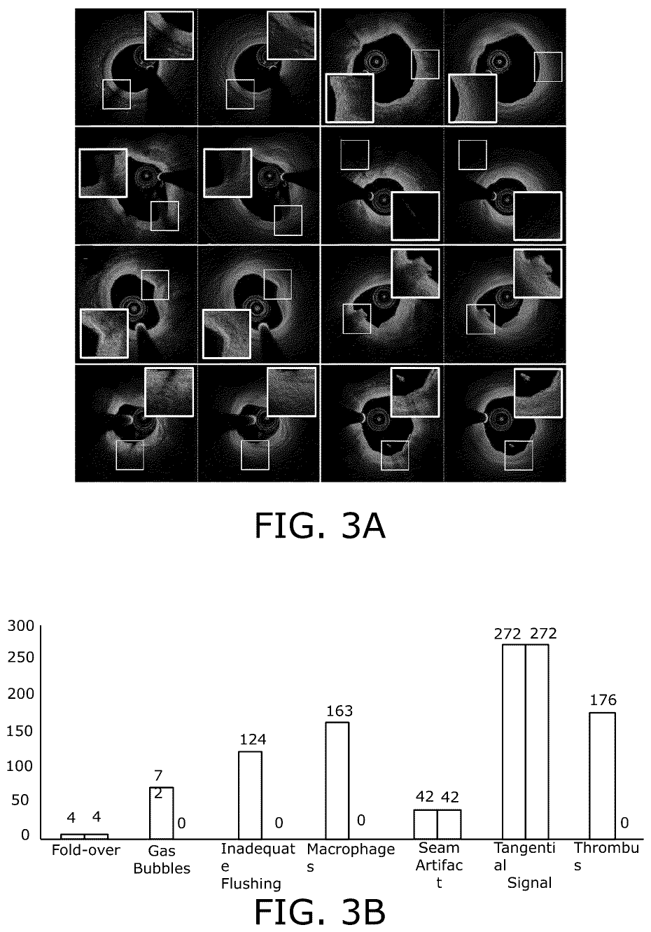

FIG. 3A provides comparison of uncorrected OCT image and artifact-corrected OCT image. In particular, FIG. 3A provides eight instances of comparisons with uncorrected OCT image (left panel) and artifact-corrected OCT image (right panel) depicting Gas Bubbles, Inadequate Flushing, Macrophages causing signal dropout, Non-uniform Rotational Distortion, Miscellaneous Shadowing, Saturation artifact, Thrombus, Microthrombus. Herein, insets are 2.0× magnification images from areas outlined. Further, FIG. 3B provides a graph depicting the effect of correction on different types of artifacts that affect clinical measurements. All frames were processed using the artifact correction techniques as proposed, with an artifact deemed corrected if it was no longer visible in the frame or no longer affected clinical measurements. 2335/3318 (70.4%) artifacts were corrected, with true tissue reconstruction achieved in 535 artifacts which would otherwise prevent accurate clinical measurements. Tissue reconstruction was achieved in all cases of thrombus, macrophage shadows, inadequate flushing, and gas bubbles. In contrast, distortion caused by tangential signal drop out, seam artifacts, and fold-over artifacts could not be corrected (as shown in FIG. 3B).

Next, the effect of correction on OCT lumen measurements, plaque classification and identification of higher risk plaque features were examined.

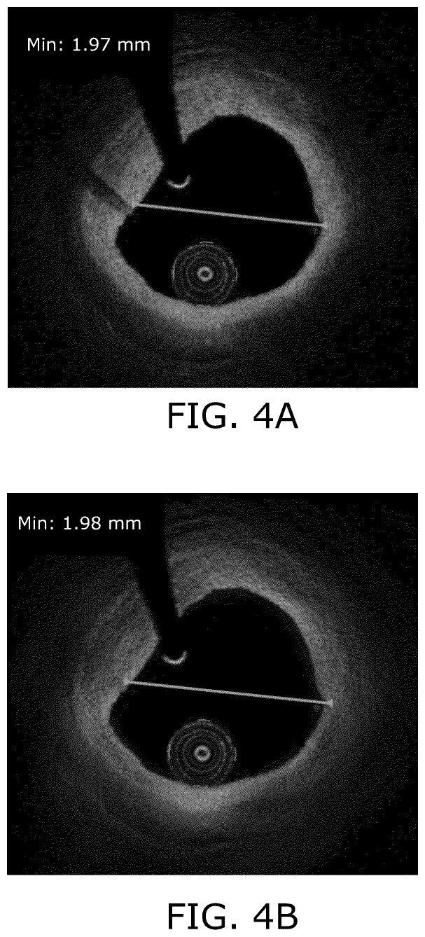

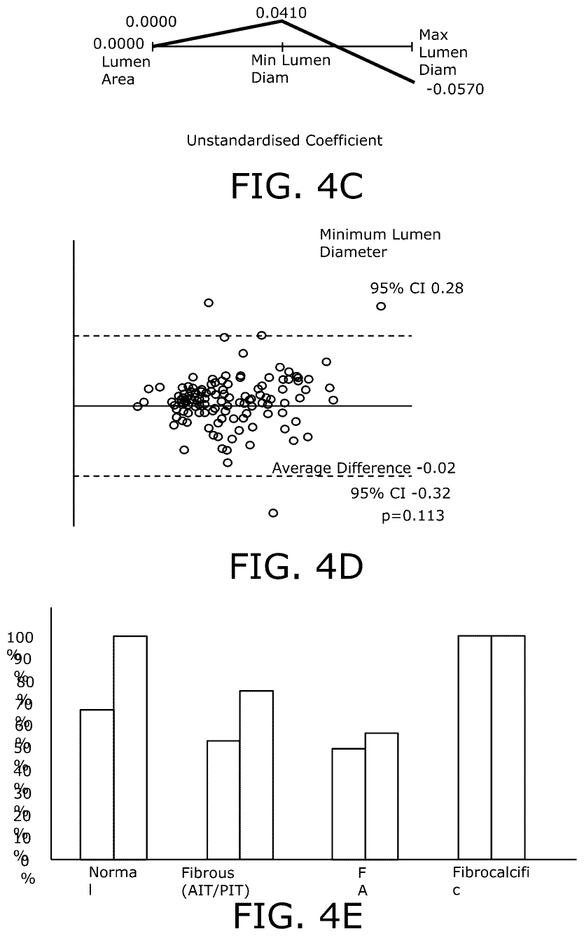

Reference to FIGS. 4A-4D is made to ensure that the correction of OCT artifacts does not alter vessel size or shape, and improves plaque classification of fibroatheromas, intimal thickening and normal vessels. Herein, FIG. 4A and FIG. 4B provides a comparison of minimum lumen diameter measurements taken on uncorrected OCT images (as shown in FIG. 4A) and artifact-corrected OCT images (as shown in FIG. 4B). Further, FIG. 4C provides correlation coefficient plots for lumen area, minimum and maximum lumen diameter measurements with unstandardised correlation coefficients indicated; and FIG. 4D provides a Bland-Altman plot showing agreement between minimum lumen diameter measurements taken on uncorrected and artifact-corrected OCT images. 95% confidence intervals (CI) are shown. All measurements were made in mm. Artifacts are denoted by an arrow. Herein, the percentage of uncorrected and artifact-corrected OCT frames that accurately classified plaques are compared against histology. The left bar corresponds to uncorrected OCT images and the right bar corresponds to artifact-corrected OCT images. Data is in means.

As depicted, the lumen areas showed excellent correlation (unstandardised coefficient 0.000) with no statistically significant difference between uncorrected and corrected OCT images (p=0.982). Maximum and minimum lumen diameters on corrected and uncorrected OCT images also showed excellent agreement (unstandardised coefficient 0.057, p=0.115, and 0.041, p=0.113 respectively) (as shown in FIG. 4A). These measurements indicate that the correction of OCT artifacts does not alter vessel size or shape.

Further, to identify the effects of correction on OCT plaque classification and higher-risk features, 113 OCT images containing artifacts were co-registered with histology images. 5 sections were excluded from final analysis due to incomplete histology owing to fragmentation and loss of plaque material. The 108 histological regions of interest (ROI) represented a range of different plaque types. ROI were classified as normal vessel (n=3, 2.8%), fibroatheroma (n=53, 49.1%), adaptive intimal thickening (n=17, 15.7%), pathological intimal thickening (n=23, 21.3%) and fibrocalcific plaque (n=12, 11.1%).

Uncorrected OCT plaque classification disagreed with histological classification in 43.5% ROI (n=47) that contained artifacts (as shown in FIG. 4B). In particular, 50.9% of fibroatheroma sections were misclassified via uncorrected OCT, and 47.5% fibrous tissue (AIT or PIT) sections. In contrast, fibrocalcific lesions and normal vessels were accurately identified in 100% and 68% uncorrected OCT artery sections respectively. Artifact correction reduced disagreement between OCT and histological classification to 30.6% overall, with fibrous tissue now correctly identified via OCT in 75% of sections (n=30). The misclassification rate for fibroatheroma reduced from 50.9% to 43.4% after artifact correction.

The observed diagnostic accuracy of plaque classification using OCT was uniformly improved after correction of images containing artifacts. The accuracy for fibroatheroma was 65.7% for uncorrected OCT and 75.0% for artifact-corrected OCT (provided in Table 2 below), with sensitivities to detect fibroatheroma of 49.1% (uncorrected) and 56.6% (artifact corrected). Accuracy of fibrous tissue detection was 73.3% with uncorrected OCT compared with 82.4% utilizing corrected OCT images. 27 of 53 fibroatheroma incorrectly identified by uncorrected OCT were classified as fibrous tissue on 9 occasions and 18 times as fibrocalcific plaque. Similarly corrected OCT misclassified fibroatheroma as fibrous tissue on 9 occasions and fibrocalcific plaque on 14 times. The incorrectly classified tissues showed several common features. For example, tissue incorrectly identified as fibroatheroma by OCT had a thick fibrous cap or large area of fibrous tissue on histology sections suggesting the ability of OCT to identify tissue is entirely dependent on penetration of light into the vessel wall. Incorrectly identified fibroatheroma frames displayed 27 artifacts overall, in contrast with falsely identified fibroatheroma with corrected OCT which showed 4 artifacts (fold-over n=1, seam artifact n=3, tangential signal drop out n=1).

TABLE 2
Accuracy of uncorrected OCT and artifact corrected OCT
plaque classification compared with histology.

| Histological Classification | | | |
|---|---|---|---|
| Normal | Fibrous | FA | Fibrocalcific |
| (n = 3) | (n = 40) | (n = 53) | (n = 12) |

Uncorrected OCT

| | | | | |
|---|---|---|---|---|
| Correctly Identified (n) | 2/3 | 21/40 | 26/53 | 12/12 |
| Sensitivity (%) | 66.7 | 70.0 | 49.1 | 100.0 |
| Specificity (%) | 93.3 | 86.8 | 81.8 | 78.1 |
| PPV (%) | 22.2 | 69.0 | 72.2 | 36.4 |
| NPV (%) | 99.0 | 75.5 | 62.5 | 100 |
| Diagnostic Accuracy (%) | 92.6 | 73.3 | 65.7 | 80.6 |

Artefact Corrected OCT

| | | | | |
|---|---|---|---|---|
| Correctly Identified (n) | 3/3 | 30/40 | 30/53 | 12/12 |
| Sensitivity (%) | 100.0 | 75.0 | 56.6 | 100.0 |
| Specificity (%) | 95.2 | 86.8 | 92.7 | 84.4 |
| PPV (%) | 37.5 | 76.9 | 88.2 | 44.4 |
| NPV (%) | 100 | 85.5 | 68.9 | 100 |
| Diagnostic Accuracy (%) | 95.4 | 82.4 | 75 | 86.1 |

As per Table 2, fibrous includes AIT and PIT; FA, fibroatheroma; NPV, negative predictive value; PPV, positive predictive value.

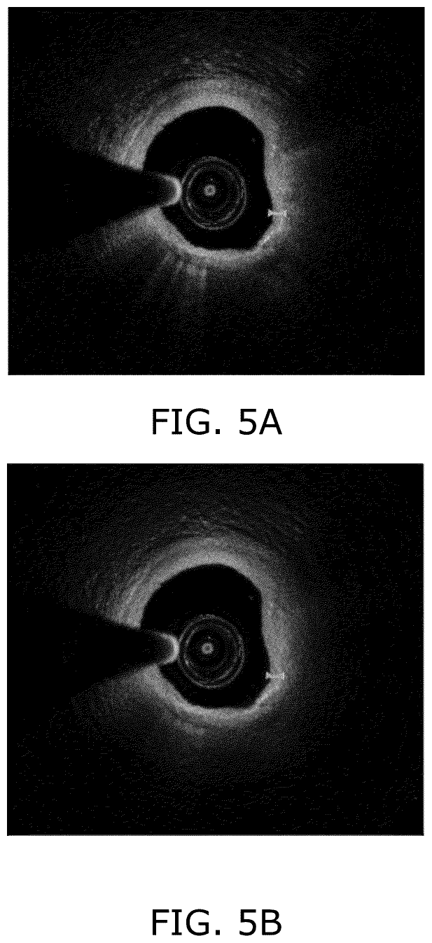

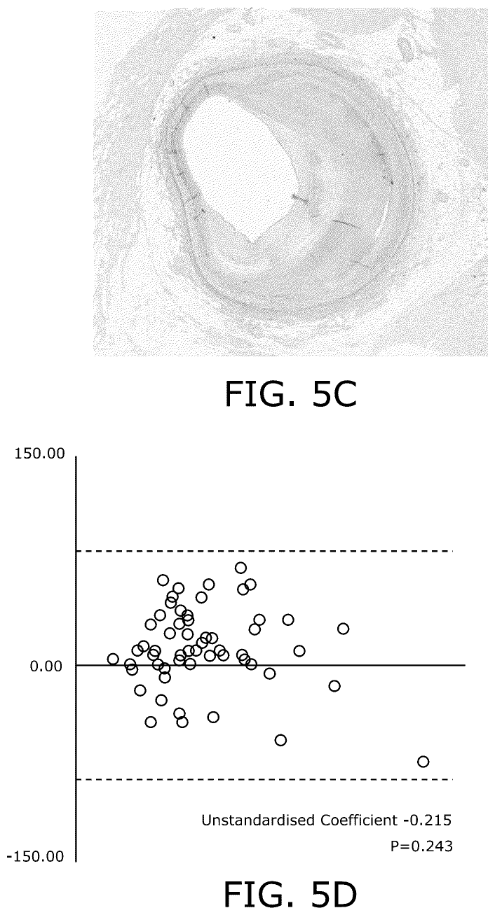

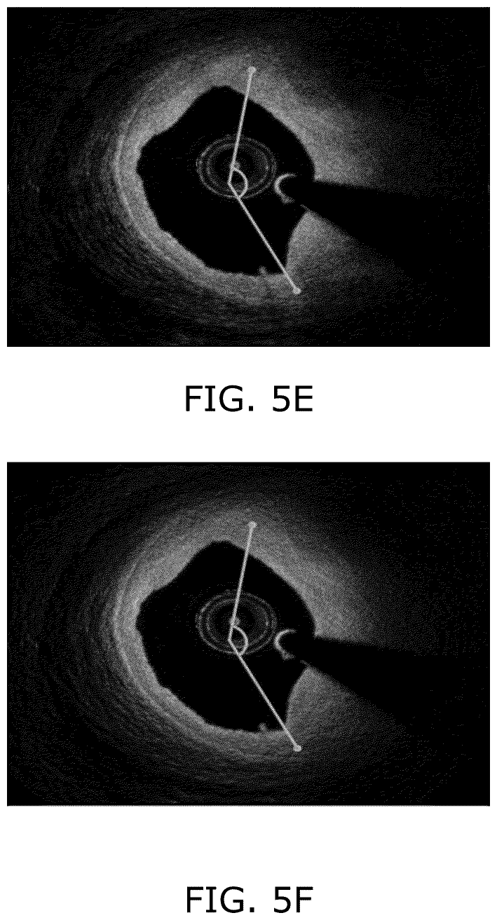

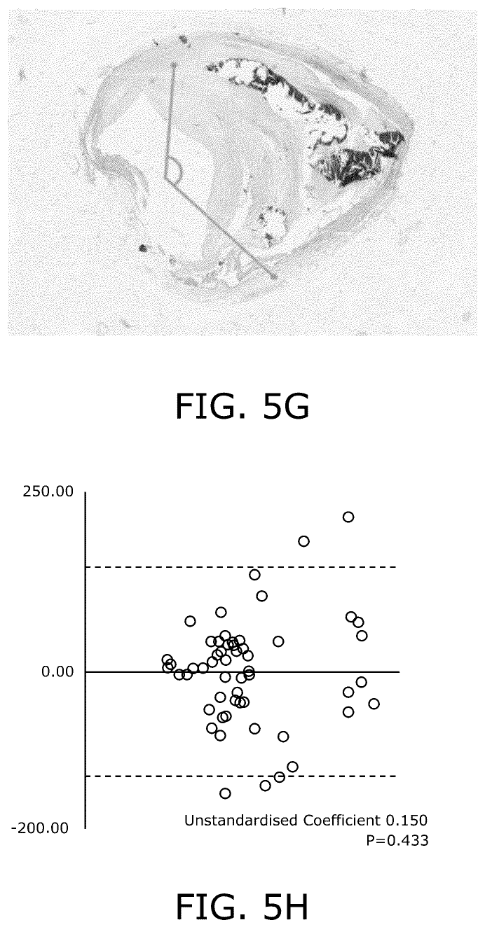

Reference to FIGS. 5A-5H is made to discuss that correction of artifacts in OCT images does not alter FCT measurements or lipid arc. Herein, FIG. 5A provides fibrous cap thickness (FCT) measured on uncorrected OCT images, FIG. 5B provides FCT measured on artifact-corrected OCT images, and FIG. 5C provides FCT measured on co-registered histology images. All measurements were made in microns. Further, FIG. 5D provides Bland-Altman plot of FCT of artifact-corrected OCT images and histology images. FIG. 5E provides a lipid arc measured on uncorrected OCT images, FIG. 5F provides lipid arc measured on artifact-corrected OCT images, and FIG. 5G provides a lipid arc measured on co-registered histology images. Again, all measurements were made in degrees. Further, FIG. 5H provides a Bland-Altman plot of artifact-corrected OCT images and histology images.

As OCT-identified thin fibrous caps and large lipid arcs are higher risk features for future MACE, examination was therefore made whether artifact correction affected FCT and lipid arc measurements, thus rendering these measurements invalid. Fibrous cap thickness (FCT) was measured three times at its thinnest point, with a mean used for final analysis. Uncorrected OCT derived FCT measurements correlated well with histology-derived measurements with no statistically significant difference (unstandardised correlation coefficient 0.079, p=0.729). Similarly, artifact corrected FCT measurements showed statistically accurate correlation with histology measurements (unstandardised correlation coefficient −0.215, p=0.243). Lipid arc measurements from uncorrected OCT frames had moderate correlation (unstandardised correlation coefficient 0.077) but were subject to proportional bias (p=0.018). In contrast, artifact-corrected lipid arc measurements showed a statistically accurate correlation without a proportional bias (unstandardised correlation coefficient 0.150, p=0.433), indicating that the correction of OCT artifacts improves the accuracy of lipid arc measurements in frames containing artifacts.

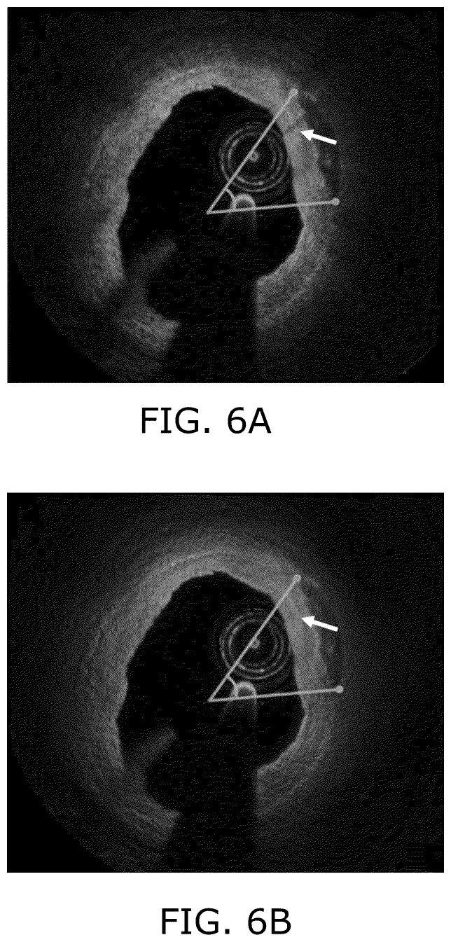

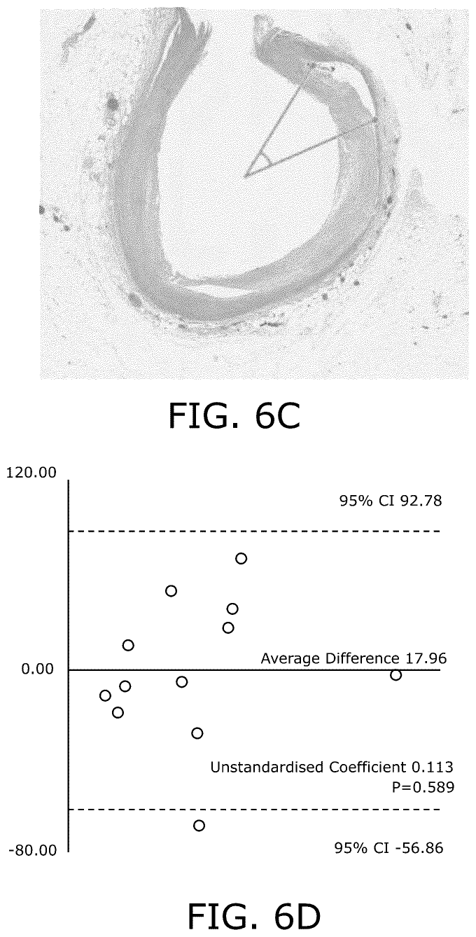

Reference to FIGS. 6A-6D is made for analysis of calcium arcs. The calcium arc is an important measurement that can guide whether a plaque should undergo stenting or additional alternative approaches, for example rotational atherectomy or intracoronary lithotripsy. Herein, FIG. 6A provides a calcium angle measured on uncorrected OCT images, FIG. 6B provides a calcium angle measured on artifact-corrected OCT images, and FIG. 6C provides a calcium angle measured on co-registered histology images. All measurements were made in degrees. Further, FIG. 6D provides a Bland-Altman plot of calcium arc measurements on corrected OCT images and histology images. As shown, both uncorrected and corrected OCT derived calcium arcs correlated well with histology, although there was a marginal performance gain with corrected OCT images (unstandardised coefficient 0.113, p=0.589 vs. 0.127, p=0.567).

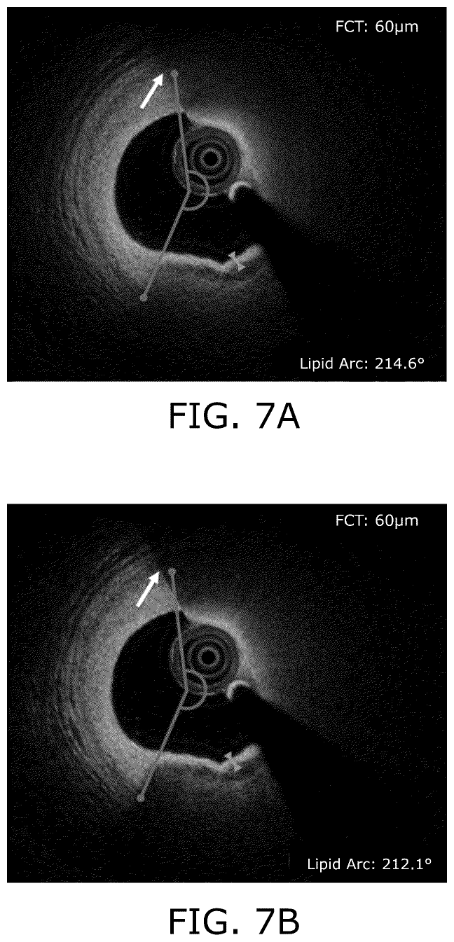

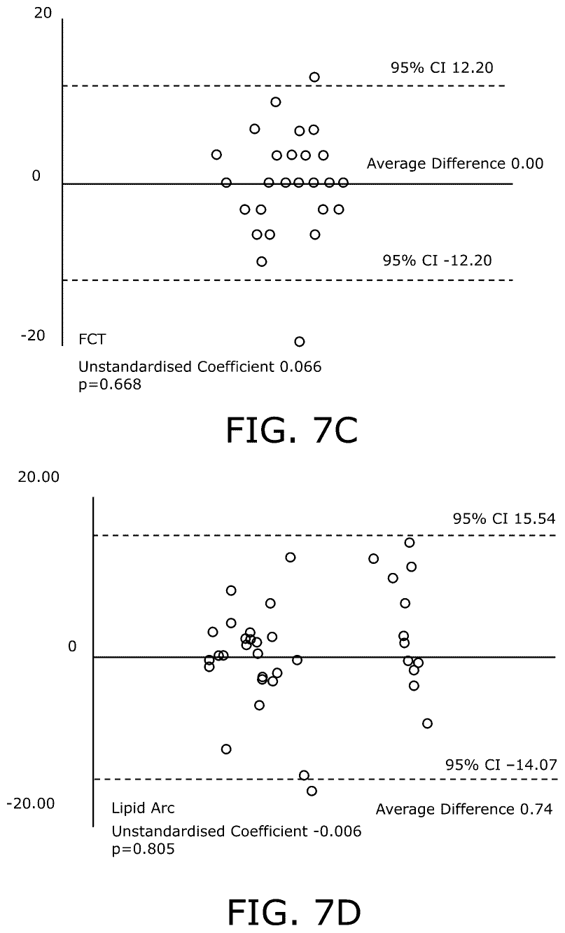

Reference to FIGS. 7A-7D is made in relation to the observation that the correction of artifacts in OCT images does not alter FCT or lipid arc measurements at TCFAs. Herein, FIG. 7A provides FCT and lipid arc measured on uncorrected OCT images, and FIG. 7B provides FCT and lipid arcs measured on artifact-corrected OCT images. All measurements were made in microns and degrees respectively. Further, FIG. 7C provides a Bland-Altman plot of FCT measurements on artifact-corrected OCT images compared with uncorrected OCT images, and FIG. 7D provides a Bland-Altman plot of lipid arc measurements on artifact-corrected OCT images compared with uncorrected OCT images. 95% confidence intervals (CI) are shown. The presence of seam artifacts is denoted by an arrow.

OCT-derived thin fibrous caps and large lipid arcs are higher risk features for future MACE. Therefore, the types of artifacts present in real world OCT defined thin-cap atheroma (TCFA) images and if these artifacts prevented accurate FCT and lipid arc measurements were examined. TCFA was defined as fibroatheroma with a fibrous cap <65 μm on 3 or more consecutive frames on uncorrected OCT(5). 43/116 (37.1%) OCT frames classified as TCFA contained an artifact, comprising macrophage shadows (n=32, 74.4%), seam artifacts (n=3, 7.0%), and tangential signal drop out (n=8, 18.6%), all of which prevented accurate measurements. Seam artifacts and tangential signal drop out could not be corrected. In contrast, all cases of macrophage shadow could be corrected, increasing measurable frames in TCFAs by 74.4%. Both corrected FCT and lipid arc measurements correlated well with uncorrected measurements with no statistically significant differences (unstandardised correlation coefficient 0.066, p=0.668 and unstandardised correlation coefficient −0.006, p=0.805 respectively), indicating that the correction of OCT artifacts does not alter clinically important measurements for high-risk plaque.

Furthermore, the results show that pre-processing (as proposed) improves the proportion of OCT frames that can accurately measure clinical features, identification of tissue types, and plaque classification ex-vivo against histology. Therefore, the proposed software's performance was subsequently tested against clinical datasets. 12 OCT pullbacks from 10 patients representing 2919 frames from the DECODE study were analyzed, which examined OCT features in patients with stable angina or NSTEMI. Patient and OCT demographics are shown in Table 3 below. Frames were classified as normal vessel (n=658, 22.5%), fibrous tissue (AIT/PIT)(n=517, 17.7%), fibroatheroma (n=685, 23.5%) and fibrocalcific plaque (n=163, 5.6%).

TABLE 3
DECODE Patient and OCT Demographics

| | Overall | SA | NSTEMI |
|---|---|---|---|
| Patient Demographics | (n = 10) | (n = 5) | (n = 5) |
| Male, n (%) | 8 (80.0) | 4 (40.0) | 4 (40.0) |
| Age, y (SD) | 59.0 (9.0) | 62.0 (14.6) | 55.0 (6.6) |

Comorbidities, n (%)

| Smoker | 5 (50.0) | 3 (60.0) | 2 (66.7) |
|---|---|---|---|
| Hypertension | 1 (10.0) | 1 (20.0) | 0 (0.0) |
| Family history of CAD | 5 (50.0) | 2 (66.7) | 3 (60.0) |
| Hyperlipidemia | 1 (10.0) | 1 (20.0) | 0 (0.0) |
| Cardiac Failure | 1 (10.0) | 1 (20.0) | 0 (0.0) |
| OCT Pullback | Overall | SA | NSTEMI |
| Demographics | (n = 12) | (n = 6) | (n = 6) |

Vessel, n (%)

| | | | |
|---|---|---|---|
| LAD | 7 (58.3) | 3 (50.0) | 4 (66.7) |
| LCx | 2 (16.7) | 2 (33.3) | 0 (0.0) |
| RCA | 3 (25.0) | 1 (16.7) | 2 (33.3) |

AHA Lesion Classification, n (%)

| | | | |
|---|---|---|---|
| A | 8 (66.7) | 5 (41.7) | 3 (25.0) |
| B | 4 (33.3) | 1 (8.3) | 3 (25.0) |
| C | 0 (0.0) | 0 (0.0) | 0 (0.0) |

Plaque Classification, frames (%)

| | | | |
|---|---|---|---|
| Normal | 658 (22.5) | 364 (23.6) | 294 (21.4) |
| Fibrous (AIT/PIT) | 517 (17.7) | 209 (14.9) | 308 (22.4) |
| FA | 685 (23.5) | 311 (20.2) | 374 (27.2) |
| Fibrocalcific | 163 (5.6) | 138 (8.9) | 25 (1.8) |

An artifact was present in 2023 out of 2919 frames (69.3%), including gas bubbles (n=218), inadequate flushing (n=1105), macrophage shadows (n=194), superficial signal drop out (n=190), saturation (n=52), seam artifacts (n=151), tangential signal drop out (n=17), and thrombus (n=96) (as shown in Table 4 below). Compared to ex-vivo OCT pullbacks, the clinical pullbacks contained comparatively more instances of inadequate flushing (54.6% vs. 15.4%) and superficial signal drop out (9.4% vs. 2.4%), but less gas bubbles (10.8% vs. 24.2%), seam artifacts (7.5% vs. 20.2%), tangential signal drop out (0.8% vs. 8.9%) and thrombus (4.7% vs. 17.5%). Overall, an artifact was over or within a lesion in 819/2023 frames (40.5%) and could prevent accurate clinical measurements in 291/2919 frames (10.0%) in a typical clinical OCT pullback.

TABLE 4
Comparison of Clinical and Ex-Vivo Artifact Frames

| | In Vivo | Ex Vivo |
|---|---|---|
| | (n = 2023) | (n = 3318) |
| Artifact Classification | Number (%) | Number (%) |
| Fold-over | 0 (0.0) | 17 (0.5) |
| Gas Bubbles | 218 (10.8) | 804 (24.2) |
| Inadequate Flushing | 1105 (54.6) | 510 (15.4) |
| Macrophage Shadow | 194 (9.6) | 325 (9.8) |
| Superficial Signal Drop out | 190 (9.4) | 79 (2.4) |
| Saturation | 52 (2.6) | 38 (1.1) |
| Seam Artifact | 151 (7.5) | 671 (20.2) |
| Tangential Signal Drop Out | 17 (0.8) | 295 (8.9) |
| Thrombus | 96 (4.7) | 579 (17.5) |

In table 4, CAD indicates coronary artery disease; Cardiac failure defined as left ventricular ejection fraction ≤50%; LAD indicates left anterior descending artery; LCx indicates left circumflex artery; RCA indicates right coronary artery; AIT indicates adaptive intimal thickening; and PIT, pathological intimal thickening.

Also, results show that artifact correction changes clinical plaque classification. 1829/2023 (90.4%) artifacts were corrected with the artifact correction software, with an artifact deemed corrected if it was no longer visible in the frame or if it no longer affected clinical measurements. True tissue reconstruction could be achieved in 244 (83.8%) artifacts which would otherwise prevent accurate clinical measurements, including all cases of gas bubbles, inadequate flushing, macrophage shadows, superficial signal drop out, and saturation artifacts. Distortion caused by tangential signal drop out and seam artifacts could not be corrected, but in contrast to the present ex-vivo work, only 72.9% of artifacts caused by thrombus could be corrected. Thrombus in these frames was larger than typically observed ex-vivo and cast a large and dense shadow across the observed tissue. Correction of artifacts changed plaque classification in 6.5% frames (n=132), particularly for fibrous tissue (n=37, 7.2%) and fibroatheroma (n=92, 13.4%), whilst classification of fibrocalcific plaques was unaffected.

Previous studies have reported high accuracy with regards to OCT's ability to identify different tissue types, therefore this aspect was also checked to establish if image processing was able to maintain a high diagnostic precision. The observed diagnostic accuracy of plaque classification using artifact corrected OCT compared with uncorrected OCT was uniformly high. The accuracy for fibroatheroma was 95.3% for artifact-corrected OCT, with sensitivities to detect fibroatheroma of 86.6%. Accuracy of fibrous tissue detection was 93.5%. Normal tissue was detected with 98.2% accuracy and fibrocalcific plaque 100% accuracy (as shown in Table 5 below).

TABLE 5
Accuracy of artifact corrected optical coherence tomographic
plaque classification compared with uncorrected OCT.

| Uncorrected OCT Classification | | | |
|---|---|---|---|
| | Fibrous | | |
| Normal | (AIT/PIT) | FA | Fibrocalcific |
| (n = 658) | (n = 517) | (n = 685) | (n = 163) |

Artifact Corrected OCT

| | | | | |
|---|---|---|---|---|
| Correctly | 655/658 | 480/517 | 593/685 | 163/163 |
| Identified (n) | | | | |
| Sensitivity (%) | 99.5 | 92.8 | 86.6 | 100.0 |
| Specificity (%) | 97.6 | 93.7 | 99.7 | 100.0 |
| PPV (%) | 92.3 | 76.0 | 98.9 | 100.0 |
| NPV (%) | 99.9 | 98.4 | 96.0 | 100.0 |
| Diagnostic | 98.2 | 93.5 | 95.3 | 100.0 |
| Accuracy (%) | | | | |

As per Table 5, fibrous includes adaptive intimal thickening and pathological intimal thickening; FA, fibroatheroma; NPV, negative predictive value; PPV, positive predictive value.

Plaque features such as thin fibrous caps and large lipid arcs are higher risk features for future MACE. However, while OCT can detect many higher risk features, the presence of artifacts inherent to different tissue types and OCT acquisition makes accurate identification and measurement difficult. Indeed, although studies have demonstrated good correlation between FCT measured on OCT images and histology images, the positive predictive value of OCT for characterizing TCFA against histology can be as low as 41%, potentially explaining discrepancies between the incidence of TCFA in OCT studies compared with histology. Micron-level accuracy for measurements is also important, as potent anti-atherosclerosis drugs show changes in FCT interpreted as promoting plaque stability of only 23 μm and decrease in lipid arc of only 12.4 degrees.

It was found that (a) Ex-vivo OCT pullbacks and those obtained from clinical studies contain a wide range of artifacts, including thrombus, macrophages, inadequate flushing, gas bubbles, fold-over artifact, seam artifact and tangential signal drop out; (b) Artifacts were present in nearly 50% of frames at post-mortem, 62.7% of these were over or within a plaque, and ˜12% prevented accurate measurement of plaque features; (c) Clinical OCT pullbacks contained a similar range and incidence (10.0%) of artifacts; (d) Image pre-processing could correct all cases of macrophage shadow, inadequate flushing, and gas bubbles, and most cases of thrombus, but not fold-over artifact, seam artifacts and tangential signal drop out; (e) Image pre-processing reduced unmeasurable frames to <5% ex-vivo and ˜1% in-vivo; (f) Ex-vivo image pre-processing improved the accuracy of fibrous tissue and fibroatheroma detection from 73.3% and 65.7%, to 82.4% and 75.0% respectively; (f) Image pre-processing did not affect lumen measurements, or higher risk features such as fibrous cap thickness or lipid arcs ex-vivo or in clinical studies.

It was also found that artifacts on OCT frames can impair its ability to identify tissue types and measurements, affecting both plaque classification and identification of higher risk features. In the present ex-vivo study, fold-over artifact (0.5%), gas bubbles (8.4%), inadequate flushing (14.5%), macrophage shadows (19.1%), seam artifacts (4.9%), tangential signal drop out (31.9%), and thrombus (20.6%) were found over or within a lesion and prevented accurate clinical assessment. A similar range of artifacts was found in the clinical analysis, with gas bubbles (1.7%), inadequate flushing (5.8%), superficial signal drop out 3.8%), seam artifacts (1.4%), tangential signal drop out (5.8%) and thrombus (15.5%) being observed, but with a significantly higher incidence of macrophage shadows (66.0%) within a lesion also being identified, and preventing accurate clinical assessment. Overall, true tissue reconstruction could be achieved in most cases of thrombus (72.9%), and all cases of macrophage shadow, inadequate flushing, and gas bubbles, but tangential signal drop out, seam artifacts, and fold-over artifacts could not be corrected. Image pre-processing reduced the proportion of frames where accurate clinical measurements could not be made to <5% ex-vivo and 1.6% in-vivo.

Although published studies report high accuracy of ex-vivo OCT for identification of plaque tissues, the initial validation studies were often performed in non-coronary arteries and with older time-domain systems. More recent studies have quoted similar high accuracy for identification of plaque types and for FCT measurements, yet the positive predictive value for identification of high-risk plaque features is low. It was shown that a significant discordance existed between OCT-identified tissue types and plaque classification against histology, particularly for fibrous tissue and fibroatheromas. Although the accuracy of detecting normal tissue was uniformly high (92.6% in uncorrected OCT and 95.4% in corrected OCT), the ability to identify fibrous tissue and fibroatheroma with uncorrected OCT was lower (73.3% and 65.7% respectively). Artifacts which prevented accurate classification or measurement were commonly due to shadows being cast on the surrounding tissue, or the absorption of the light source leading the signal to drop out on the vessel wall. For example, macrophages strongly scatter light leading to superficial shadowing on the arterial wall which gives the appearance of a lipid-rich plaque or a thin-cap fibroatheroma. In contrast, image pre-processing improved the diagnostic accuracy to detect fibrous tissue and fibroatheroma, whilst also improving sensitivity to detect fibrous tissue and fibroatheromas.

In addition to plaque features, an MLA, <3.5 mm and high plaque burden are associated with increased MACE and poor clinical outcomes (19, 30). Importantly, image pre-processing does not significantly alter lumen area, and maximum and minimum lumen diameters. Image pre-processing also did not affect the accuracy of fibrous cap thickness or lipid arc measurements compared with histology measurements, indicating that the correction of OCT artifacts does not alter clinically important measurements of plaque or coronary lumen.

The OCT outputs may be inserted into regular processing pathways, and will provide more frames to analyze and more accurate measurements, particularly in higher-risk lesions. This accuracy is particularly important for drug studies where small changes in FCT and change in phenotype of plaques are being used to indicate its success or failure to affect plaque stability.

Thus, the teachings of the present disclosure are applicable to real world OCT use in which nearly 50% of OCT frames contain an artifact in whole pullbacks, preventing the accurate characterisation of plaque or measurements of plaque features in up to 12% of frames. Image pre-processing with artifact correction is non-destructive and improves diagnostic accuracy of OCT to discriminate plaque types without altering the measurements of vessel size or shape or clinically relevant measurements such as minimum FCT and lipid arc. Thus, the proposed image pre-processing method and system should be considered for both clinical and research use of OCT.

### DETAILED DESCRIPTION OF THE DRAWINGS

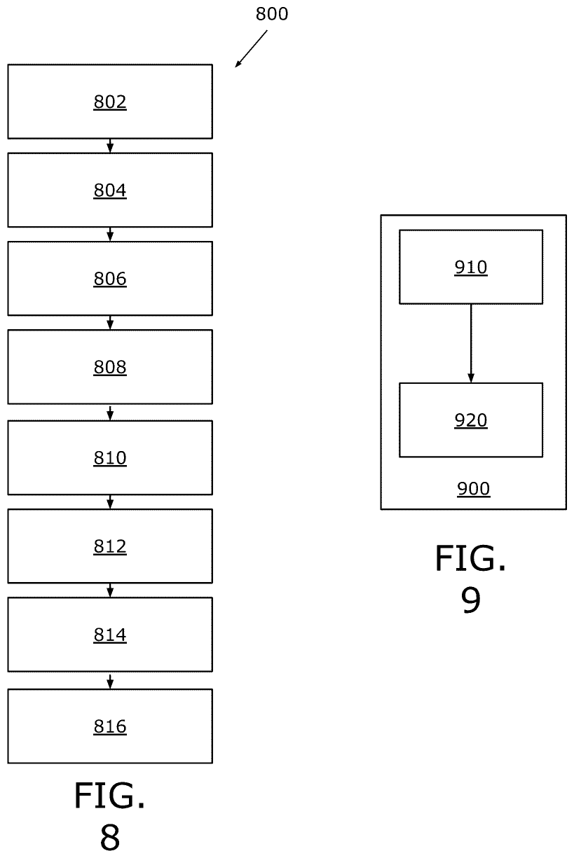

Referring to FIG. 8, illustrated is a flowchart listing steps involved in a method 800 for correction of artifacts in Optical Coherence Tomography (OCT) images, in accordance with an embodiment of the present disclosure. At step 802, the method 800 includes acquiring a given OCT image. At step 804, the method 800 includes transforming, by polar reconstruction, the given OCT image from a Cartesian coordinate system to a polar coordinate system, to generate a reconstructed polar OCT image. At step 806, the method 800 includes splitting the reconstructed polar OCT image into a Red color channel, a Green color channel and a Blue color channel, to generate corresponding polar coordinate images. At step 808, the method 800 includes performing a Fourier transform on each of the Red color channel, the Green color channel and the Blue color channel in the corresponding polar coordinate images, to convert the corresponding polar coordinate images to their respective frequency domain images. At step 810, the method 800 includes applying a custom frequency mask to each of the frequency domain images to filter out frequencies corresponding to one or more artifacts, to generate respective corrected frequency domain images. At step 812, the method 800 includes performing an inverse Fourier transform on each of the corrected frequency domain images, to generate respective output images in the Cartesian coordinate system, with one output image for each of the Red color channel, the Green color channel and the Blue color channel. At step 814, the method 800 includes combining the output images, from each of the Red color channel, the Green color channel and the Blue color channel, to generate a reconstituted OCT image. At step 816, the method 800 includes merging the reconstituted OCT image with the given OCT image, to generate an artifact-corrected OCT image.

It may be appreciated that the above steps 802-816 are only illustrative and other alternatives can also be provided where one or more steps are added, one or more steps are removed, or one or more steps are provided in a different sequence without departing from the spirit and the scope of the present disclosure.

Referring to FIG. 9, illustrated is a schematic block diagram of a system (as represented by reference numeral 900) for correction of artifacts in Optical Coherence Tomography (OCT) images, in accordance with an embodiment of the present disclosure. The system 900 includes an input module 910 configured to acquire a given OCT image. The system 900 further includes a processing module 920 in signal communication with the input module 910. Herein, the processing module 920 is configured to: transform, by polar reconstruction, the given OCT image from a Cartesian coordinate system to a polar coordinate system, to generate a reconstructed polar OCT image; split the reconstructed polar OCT image into a Red color channel, a Green color channel and a Blue color channel, to generate corresponding polar coordinate images; perform a Fourier transform on each of the Red color channel, the Green color channel and the Blue color channel in the corresponding polar coordinate images, to convert the corresponding polar coordinate images to respective frequency domain images; apply a custom frequency mask to each of the frequency domain images to filter out frequencies corresponding to one or more artifacts, to generate respective corrected frequency domain images; perform an inverse Fourier transform on each of the corrected frequency domain images, to generate respective output images in the Cartesian coordinate system, with one output image for each of the Red color channel, the Green color channel and the Blue color channel; combine the output images, from each of the Red color channel, the Green color channel and the Blue color channel, to generate a reconstituted OCT image; and merge the reconstituted OCT image with the given OCT image, to generate an artifact-corrected OCT image.

Modifications to the embodiments of the present disclosure described in the foregoing are possible without departing from the scope of the present disclosure as defined by the accompanying claims. Expressions such as “including”, “comprising”, “incorporating”, “have”, “is” are used to describe and claim that the present disclosure is intended to be construed in a non-exclusive manner, namely allowing for items, components or elements not explicitly described also to be present. Reference to the singular is also to be construed to relate to the plural.

1. A method (800) for the correction of artifacts in Optical Coherence Tomography (OCT) images, the method (800) comprising:
acquiring a given OCT image;
transforming, by polar reconstruction, the given OCT image from a Cartesian coordinate system (900) to a polar coordinate system (900), to generate a reconstructed polar OCT image;
splitting the reconstructed polar OCT image into a Red color channel, a Green color channel and a Blue color channel, to generate corresponding polar coordinate images;
performing a Fourier transform on each of the Red color channel, the Green color channel and the Blue color channel in the corresponding polar coordinate images, to convert the corresponding polar coordinate images to respective frequency domain images;
applying a custom frequency mask to each of the frequency domain images to filter out frequencies corresponding to one or more artifacts, to generate respective corrected frequency domain images;
performing an inverse Fourier transform on each of the corrected frequency domain images, to generate respective output images in the Cartesian coordinate system (900), with one output image for each of the Red color channel, the Green color channel and the Blue color channel;
combining the output images, from each of the Red color channel, the Green color channel and the Blue color channel, to generate a reconstituted OCT image; and
merging the reconstituted OCT image with the given OCT image, to generate an artifact-corrected OCT image.

2. A method (800) according to claim 1, wherein the given OCT image is of a coronary artery.

3. A method (800) according to claim 2 further comprising analyzing the OCT image, before transforming the given OCT image, to detect one or more of a vessel lumen and a guidewire shadow therein by implementing a neural network.

4. A method (800) according to claim 3, wherein the step of merging the reconstituted OCT image with the given OCT image further comprises merging the detected one or more of the vessel lumen and the guidewire shadow, to generate the artifact-corrected OCT image.

5. A method (800) according to any of claims 3 or 4, wherein the neural network is a deep convolutional network with an encoding-decoding architecture, and wherein the neural network is trained to detect the vessel lumen and/or the guidewire shadow in the OCT images.

6. A method (800) according to any of preceding claims, wherein the custom frequency mask is configured to utilize frequency filtering to alter the magnitude of pixels corresponding to the artifacts.

7. A method (800) according to any of preceding claims, wherein the artifact-corrected OCT image comprises corrected artifacts selected from a group consisting of thrombus, macrophage shadows, inadequate flushing, and gas bubbles.

8. A system (900) for the correction of artifacts in Optical Coherence Tomography (OCT) images, the method (800) comprising:
an input module (910) configured to acquire a given OCT image; and
a processing module (920) in signal communication with the input module (910), the processing module (920) configured to:
transform, by polar reconstruction, the given OCT image from a Cartesian coordinate system (900) to a polar coordinate system (900), to generate a reconstructed polar OCT image;
split the reconstructed polar OCT image into a Red color channel, a Green color channel and a Blue color channel, to generate corresponding polar coordinate images;
perform a Fourier transform on each of the Red color channel, the Green color channel and the Blue color channel in the corresponding polar coordinate images, to convert the corresponding polar coordinate images to respective frequency domain images;
apply a custom frequency mask to each of the frequency domain images to filter out frequencies corresponding to one or more artifacts, to generate respective corrected frequency domain images;
perform an inverse Fourier transform on each of the corrected frequency domain images, to generate respective output images in the Cartesian coordinate system (900), with one output image for each of the Red color channel, the Green color channel and the Blue color channel;
combine the output images, from each of the Red color channel, the Green color channel and the Blue color channel, to generate a reconstituted OCT image; and
merge the reconstituted OCT image with the given OCT image, to generate an artifact-corrected OCT image.

9. A system (900) according to claim 8, wherein the given OCT image is of a coronary artery.

10. A system (900) according to claim 9 further comprising a neural network implemented by the processing module (920), wherein the neural network is further configured to analyze the OCT image, before transforming the given OCT image, to detect one or more of a vessel lumen and a guidewire shadow therein.

11. A system (900) according to claim 10, wherein the processing module (920), for merging the reconstituted OCT image with the given OCT image, is further configured to merge the detected one or more of the vessel lumen and the guidewire shadow, to generate the artifact-corrected OCT image.

12. A system (900) according to any of claims 10 or 11, wherein the neural network is a deep convolutional network with an encoding-decoding architecture, and wherein the neural network is trained to detect the vessel lumen and/or the guidewire shadow in the OCT images.

13. A system (900) according to any of claims 8-12, wherein the custom frequency mask is configured to utilize frequency filtering to alter the magnitude of pixels corresponding to the artifacts.

14. An apparatus comprising a computer program stored in a memory, the computer program being configured to control the apparatus to perform the method (800) according to any one of claims 1-8.

15. A computer program comprising computer executable program code, when executed the program code controls a computer to perform the method (800) according to any one of claims 1-8.
